(19) United States
(12) Patent Application Publication
(10) Pub. No.: US 2024/0179021 A1
(43) Pub. Date: May 30, 2024

(54) Methods and Systems for Enabling Electronic Access to Medical Record Information of a Veterinary Patient over a Network

(71) Applicant: IDEXX Laboratories, Inc., Westbrook, ME (US)

(72) Inventors: Anestes Fotiades, Portland, ME (US); David Kincaid, Eau Claire, WI (US); Dwayne Paschall, Frisco, TX (US); Narathip Reamaroon, Seattle, WA (US); Terry Schutte, Eau Claire, WI (US); Patricia Minerich, Boothbay, ME (US)

(21) Appl. No.: 18/521,641

(22) Filed: Nov. 28, 2023

Related U.S. Application Data
(60) Provisional application No. 63/428,992, filed on Nov. 30, 2022.

(51) Int. Cl. H04L 9/00

(52) U.S. Cl. CPC H04L 9/50

(57) ABSTRACT

In an example, a method includes receiving an input from a first computer system including medical record information associated with a veterinary patient and a veterinary patient identifier unique to the veterinary patient, saving the medical record information to a second computer system different from the first computer system, updating a distributed ledger to associate the veterinary patient identifier with the medical record information saved to the second computer system using a cryptographic hash pointing to a data block of the distributed ledger, receiving an access request from a third computer system different from the first computer system and the second computer system, accessing the distributed ledger to retrieve an updated cryptographic hash, and based on the updated cryptographic hash, electronically enabling access by the third computer system to the medical record information of the veterinary patient saved at the second computer system.

### CROSS REFERENCE TO RELATED APPLICATION

The present application claims priority to U.S. Provisional Application No. 63/428,992, filed on Nov. 30, 2022, the entire contents of which are herein incorporated by reference.

### FIELD

The present disclosure relates generally to methods and systems for enabling electronic access to medical record information of a veterinary patient over a network, and more particularly, to generating a universal veterinary patient identifier and unified medical records for veterinary patients.

### BACKGROUND

Veterinary records for veterinary patients are routinely kept at a veterinary office, and include details of office visits such as vital statistics, symptoms, suspected diagnosis, treatment, billing, and accompanying notes. When a veterinary patient visits multiple veterinary offices, however, records are not connected across different computing platforms and veterinary practices of the different veterinary offices. As such, often times, it is difficult to obtain complete and/or accurate veterinary patient records.

In addition, when a veterinary patient visits a veterinary office, a veterinary patient identifier is manually entered at a point-of-care office. As the veterinary patient visits different veterinarians or different veterinary offices, new or different veterinary patient identifiers are created for the veterinary patient specific to the point-of-care office. Thus, transferring veterinary patient records is difficult or impossible due to lack of a mechanism for associating records to the veterinary patient, resulting in incomplete documentation of a medical history for the veterinary patient.

Accordingly, a more effective system is needed for providing veterinarians and laboratory technicians with access to veterinary patient records across unconnected or discrete veterinary offices.

### SUMMARY

In an example, a computer-implemented method for enabling electronic access to medical record information of a veterinary patient over a network is described. The method comprises receiving, at a server, an input from a first computer system, and the input comprises medical record information associated with a veterinary patient and a veterinary patient identifier unique to the veterinary patient. The method also comprises saving the medical record information to a second computer system different from the first computer system, updating a distributed ledger to associate the veterinary patient identifier with the medical record information saved to the second computer system using a cryptographic hash pointing to a data block of the distributed ledger, and receiving, at the server, an access request from a third computer system different from the first computer system and the second computer system and the access request specifying the veterinary patient identifier. The method also comprises accessing the distributed ledger, using the veterinary patient identifier, to retrieve an updated cryptographic hash, and based on the updated cryptographic hash, the server electronically enabling access by the third computer system to the medical record information of the veterinary patient saved at the second computer system.

In another example, a server is described comprising one or more processors, and non-transitory computer readable medium having stored therein instructions that when executed by the one or more processors, causes the server to perform functions. The functions comprise receiving an input from a first computer system and the input comprises medical record information associated with a veterinary patient and a veterinary patient identifier unique to the veterinary patient, and saving the medical record information to a second computer system different from the first computer system. The functions also comprise updating a distributed ledger to associate the veterinary patient identifier with the medical record information saved to the second computer system using a cryptographic hash pointing to a data block of the distributed ledger, receiving an access request from a third computer system different from the first computer system and the second computer system and the access request specifying the veterinary patient identifier, and accessing the distributed ledger, using the veterinary patient identifier, to retrieve an updated cryptographic hash. The functions also comprise based on the updated cryptographic hash, the server electronically enabling access by the third computer system to the medical record information of the veterinary patient saved at the second computer system.

In another example, a non-transitory computer readable medium is described having stored thereon instructions, that when executed by one or more processors of a computing device, cause the computing device to perform functions. The functions comprise receiving an input from a first computer system and the input comprising medical record information associated with a veterinary patient and a veterinary patient identifier unique to the veterinary patient, saving the medical record information to a second computer system different from the first computer system, and updating a distributed ledger to associate the veterinary patient identifier with the medical record information saved to the second computer system using a cryptographic hash pointing to a data block of the distributed ledger. The functions also comprise receiving an access request from a third computer system different from the first computer system and the second computer system, the access request specifying the veterinary patient identifier, accessing the distributed ledger, using the veterinary patient identifier, to retrieve an updated cryptographic hash, and based on the updated cryptographic hash, the server electronically enabling access by the third computer system to the medical record information of the veterinary patient saved at the second computer system.

The features, functions, and advantages that have been discussed can be achieved independently in various examples or may be combined in yet other examples. Further details of the examples can be seen with reference to the following description and drawings.

### BRIEF DESCRIPTION OF THE FIGURES

Examples and descriptions of the present disclosure will be readily understood by reference to the following detailed description of illustrative examples when read in conjunction with the accompanying drawings, wherein:

FIG. 1 illustrates an example of a system, according to an example implementation.

FIG. 2 illustrates an example of a server of the system FIG. 1, according to an example implementation.

FIG. 3 is an example message diagram illustrating messaging of the system of FIG. 1 to perform functions of methods described herein, according to one example implementation.

FIG. 4 is another example message diagram illustrating messaging of the system of FIG. 1 to perform functions of methods described herein, according to one example implementation.

FIG. 5 is another example message diagram illustrating messaging of the system of FIG. 1 to perform functions of methods described herein, according to one example implementation.

FIG. 6 is another example message diagram illustrating messaging of the system of FIG. 1 to perform functions of methods described herein, according to one example implementation.

FIG. 7 shows a flowchart of an example of a method for enabling electronic access to medical record information of a veterinary patient over a network, according to an example implementation.

### DETAILED DESCRIPTION

Examples of the present disclosure will now be described more fully hereinafter with reference to the accompanying drawings. Several different examples are described and should not be construed as limited to all possible alternatives. Rather, these examples are described so that this disclosure is thorough and complete and fully conveys a scope of the disclosure to those skilled in the art.

Within examples, computer-implemented methods for enabling electronic access to medical record information of a veterinary patient over a network are described. The methods include implementation of universal veterinary patient identifier accessible from disparate computer systems utilizing a blockchain network (i.e., network tasked with a shared, immutable ledger that facilitates a process of recording transactions and tracking assets). Once accessed, the veterinary patient identifier provides access to all saved medical record information associated with the veterinary patient identifier that can include a complete medical history of a veterinary patient.

Some existing solutions for correlating medical records for the same veterinary patient use unreliable algorithms or token-based solutions that are difficult to carry across different computer systems where customers are given a “key” containing a unique ID utilized across systems. But, such methods require knowledge of the key by all systems requesting access causing inherent unavailable of permissions being provided.

The systems and methods described herein provide a solution to enable computing devices to update a distributed ledger to associate the veterinary patient identifier with all medical record information saved at a computer system using a cryptographic hash pointing to a data block of the distributed ledger, and then upon receiving an access request from a further computer system, the distributed ledger is accessed to retrieve an updated cryptographic hash that is used to electronically enable access by the further computer system to the medical record information of the veterinary patient.

Implementations of this disclosure provide technological improvements that are particular to computer technology, for example, those concerning enabling access to saved electronic data across computer networks. Computer-specific technological problems, such as associating saved electronic data across multiple veterinary clinics or offices with a universal veterinary patient identifier, and using the blockchain to manage the saved transactions with a cryptographic hash for access to the data, are solved by the methods described herein. For example, implementation of embodiments described in this disclosure allows for broad access to electronic medical history information of veterinary patients by computing systems within different veterinary clinics unassociated with each other. Thus, diagnostic data and medical records for the same veterinary patient, regardless of where the diagnostic data and medical record was created, are centrally stored and associated with the veterinary patient-in contrast to stored and associated with the veterinary clinic.

The blockchain enables an accessible link to the stored records to be retrieved upon validation of an authenticated veterinary patient identifier. The blockchain network thus holds a pointer or password to the “off-site” storage of consolidated medical records for the veterinary patient. The cryptographic hash used to document a saved transaction acts as the password to access the medical records.

In contrast to human electronic medical records (EMR), there does not exist an universally accepted identifier for veterinary patients (in contrast to a social security number for humans). In addition, human EMRs are stored on internal databases accessible through permission based requests. The disclosed systems and methods provide a solution to store medical record information and medical history data in an electronic format in a password protected data storage off-site from veterinary clinics, and access to the information is made available by retrieving the updated cryptographic hash from the blockchain. Thus, an intermediary step is required in which a universal veterinary patient identifier links to the blockchain, and then the retrieved updated cryptographic hash is used to access the medical data.

Implementations of this disclosure can thus introduce new and efficient improvements in the ways in which the blockchain is used to store and manage the universal veterinary patient identifier, which enables broad access to an associated veterinary patient's records. The blockchain allows the universal veterinary patient identifier to be used to access the medical records across a number of different environments such that medical records can be aggregated from among many different sources. A veterinarian can access a summary of medical records of all clinics to which the veterinary patient has visited as if all visits had occurred within a virtual clinic.

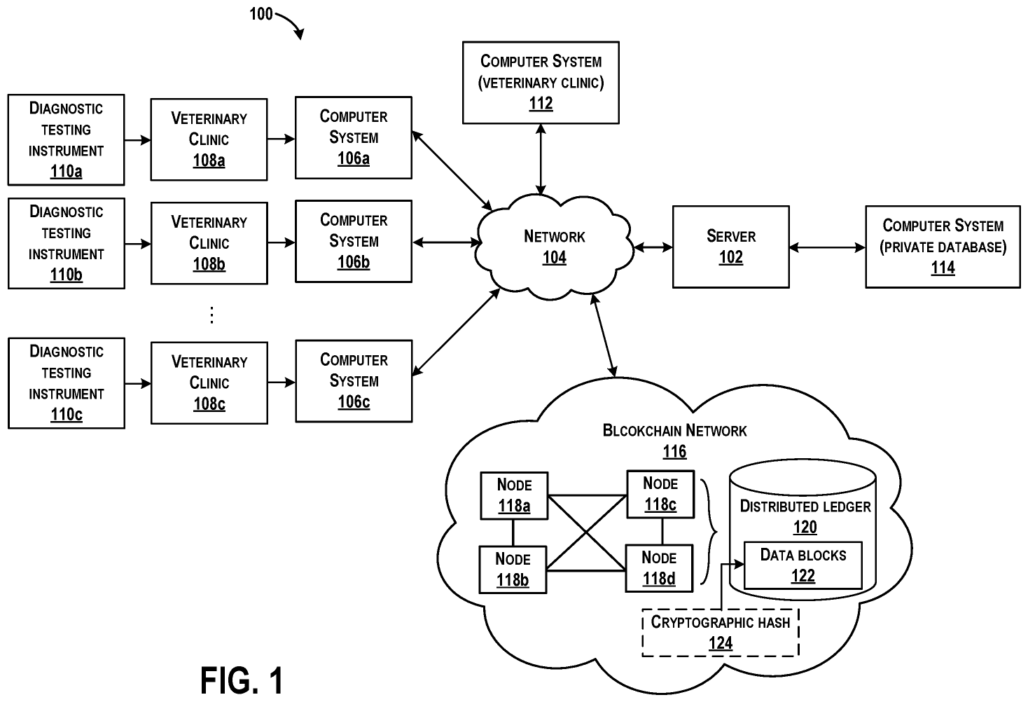

Referring now to the figures, FIG. 1 illustrates an example of a system 100, according to an example implementation. The system 100 includes a server 102 accessible through a network 104 by multiple different computer systems. One set of the computer systems includes multiple computer systems 106a-c residing at different veterinary clinics 108a-c that perform diagnostic testing of veterinary patients using diagnostic testing instruments 110a-c, for example. While three veterinary clinics 108a-c and three respective computer systems 106a-c are depicted in FIG. 1, it should be understood that this is merely an example, and systems according to the present disclosure can include any suitable number of veterinary clinics and associated computer systems. As referred to herein, the term “veterinary clinics” includes any entity at which non-human animals receive medical care, and can include brick and mortar locations, mobile clinics, on-line virtual clinics, pop-up clinics, and the like. Another computer system includes a different computer system 112 at yet a further veterinary clinic seeking access to medical record information of a veterinary patient. The server 102 stores all medical record information of veterinary patients off-site in yet another computer system 114 that includes a privately accessible database.

In the system 100, the network 104 (e.g., Internet) provides access to the server 102 for all network-connected components. In some examples, more components of the system 100 may be in communication with the network 104 to access the server 102, such as the computer system 114.

The computer systems 106-c each may be present at different veterinary clinics 108a-c residing at different geographic locations receive data (either through wired or wireless communication) with the diagnostic testing instruments 110a-c. While the example depicted in FIG. 1 includes three diagnostic testing instruments 110a-c, it should be understood that this is merely an example, and embodiments according to the present disclosure can include any suitable number of diagnostic testing instruments associated with the veterinary clinics 108a-c. Examples of the diagnostic testing instruments 110a-c include any one or combination of veterinary analyzers operable to conduct a diagnostic test of a sample of a patient (e.g., operable to determine hemoglobin amounts in a blood sample, operable to analyze a urine sample, and/or the like). Such veterinary analyzers include, for example and without limitation, a clinical chemistry analyzer, a hematology analyzer, a urine analyzer, an immunoassay reader, a sediment analyzer, a blood analyzer, a digital radiology machine, and/or the like. In one example, the computing system 106a is in communication with a veterinary analyzer of the diagnostic testing instrument 110a and is operable to receive diagnostic information from veterinary analyzer. The diagnostic testing instruments 110a-c output signals, such as signals indicative of diagnostic test results or other information, to the computing systems 106a-c.

In FIG. 1, in an example where the veterinary clinics 108a-c are physical locations, each of the veterinary clinics 108a-c resides at different location from each other and communicate medical record information (such as diagnostic information) for specific veterinary patients to the server 102 over the network 104. In another example where the veterinary clinics 108a-c are on-line virtual clinics, each of the veterinary clinics 108a-c is associated with a different entity or service provider and communicate medical record information for specific veterinary patients to the server 102 over the network 104. Thus, the server 102 receives an input from any or all of the computer systems 106a-c over time, and the input includes medical record information associated with a veterinary patient and a veterinary patient identifier unique to the veterinary patient. The server 102 saves the medical record information to the computer system 114 in the privately accessible database. The computer system 114 is different from the computer systems 106a-c and is located separately from the computer systems 106a-c.

The system 100 further includes a blockchain network 116 with nodes 118a-d that share and manage a distributed ledger 120 including data blocks 122. The blockchain network 116 is a distributed network of nodes (although only four nodes are illustrated, more or fewer may be included) that manage the distributed ledger 120 and store information electronically in the data blocks 122. The blockchain network 116 enables a secure and decentralized record of electronic transactions that have occurred on the data blocks 122. The blockchain network 116 collects information together in groups, known as the data blocks 122, which hold sets of information. The data blocks 122 have certain storage capacities and, when filled, are closed and linked to a previously filled data block, forming a chain of data known as the blockchain. All new information that follows that newly added data block is compiled into a newly formed data block that will then also be added to the chain once filled.

For comparison, a database usually structures its data into tables, whereas a blockchain, as its name implies, structures its data into chunks (i.e., data blocks) that are strung together. This data structure inherently makes an irreversible timeline of data when implemented in a decentralized nature. When a data block is filled, the data block is set in stone and becomes a part of this timeline. Each data block in the chain is given an exact timestamp when it is added to the chain.

Thus, new data blocks are always stored linearly and chronologically. That is, the new data blocks are always added to the “end” of the blockchain. After a block has been added to the end of the blockchain, it is extremely difficult to go back and alter contents of the data block unless a majority of the network has reached a consensus to do so because each data block contains its own cryptographic hash 124, along with the hash of the block before it, as well as the previously mentioned timestamp. Cryptographic hash codes are created by a mathematical function that turns digital information into a string of numbers and letters. If that information is edited in any way, then the hash code changes as well.

The blockchain network 116 allows digital information to be recorded and distributed, but not edited. In this way, the blockchain network 116 is a foundation for immutable ledgers, or records of transactions that cannot be altered, deleted, or destroyed.

In the system 100, the blockchain network 116 functions to record transactions that have occurred at the computer system 114 of the private database. Particularly, the blockchain network 116 makes a record of transactions involving saving medical record information of a veterinary patient associated with a veterinary patient identifier. Once a transaction is recorded, authenticity of the transaction is verified by the blockchain network 116. The nodes 118a-d confirm that details of the transaction are correct. After validation, the transaction is added to the data blocks 122. As mentioned, each data block on the blockchain contains its own unique hash, along with the unique hash of the block before it. When the information on a data block is edited in any way, the hash code of the data block changes-however, the hash code on the block after it would not. This discrepancy makes it extremely difficult for information on the blockchain to be changed without notice.

Thus, the server 102 saves the medical record information of the veterinary patient associated with the veterinary patient identifier, as received from any one or more of the computer systems 106a-c, to the computer system 114. Then, the server 102 accesses the blockchain network 116 to update the distributed ledger 120 and record the transaction by associating the veterinary patient identifier with the medical record information saved to the computer system 114 using the cryptographic hash 124 pointing to the data block 122 of the distributed ledger 120.

The distributed ledger 120 will thus contain the data blocks 122 that inform of a complete record of electronic transactions performed by the server 102 to save medical record information of a veterinary patient associated with the veterinary patient identifier at the computer system 114 including the privately accessible database.

The system 100 then enables electronic access to the compiled medical record information for the veterinary patient over the network 104. For example, another computer system including the different computer system 114 at yet a further veterinary clinic can request access to the medical record information for the veterinary patient. Upon receiving an access request from the computer system 112 that specifies the veterinary patient identifier, the server 102 accesses the distributed ledger 120 of the blockchain network 116, using the veterinary patient identifier, to retrieve an updated cryptographic hash of the data blocks 122 associated with the veterinary patient identifier. The cryptographic hash 124 is essentially a pointer to the medical record information stored “off-site” in the computer system 114 including the private database. Upon receiving the updated cryptographic hash, the server 102 electronically enables access by the computer system 112 to the medical record information of the veterinary patient saved at the computer system 114 via the network 104. Thus, the cryptographic hash 124 operates as permission to access the veterinary records, and can be utilized as a password to access the private database. Each time the computer system 114 private database is updated, the server 102 accesses the blockchain network 116 to update the cryptographic hash 124, and then the password to access the computer system 114 is updated as well.

In FIG. 1, in one example, the blockchain network 116 is a public blockchain, in which the public blockchain is a decentralized network with open access rights. In another example, the blockchain network 116 is a private blockchain, in which the private blockchain is a permission-based network accessible by invitation.

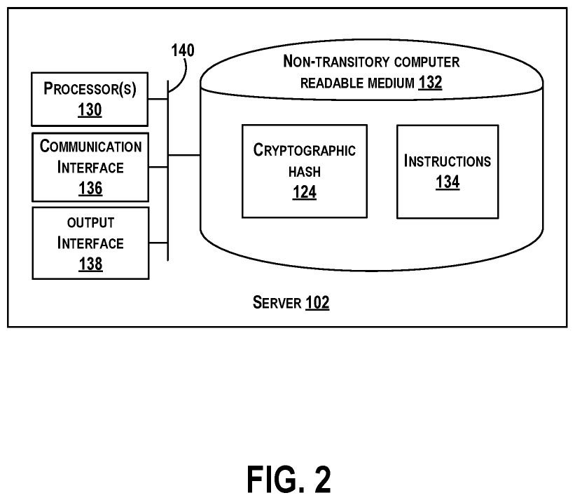

FIG. 2 illustrates an example of the server 102, according to an example implementation. The server 102 includes one or more processor(s) 130, and non-transitory computer readable medium 132 having stored therein instructions 134 that when executed by the one or more processor(s) 130, causes the server 102 to perform functions for enabling electronic access to medical record information of a veterinary patient over a network.

To perform these functions, the server 102 also includes a communication interface 136, an output interface 138, and each component of the server 102 is connected to a communication bus 140. The server 102 may also include hardware to enable communication within the server 102 and between the server 102 and other devices (not shown). The hardware may include transmitters, receivers, and antennas, for example. The server 102 may further include a display (not shown).

The communication interface 136 may be a wireless interface and/or one or more wireline interfaces that allow for both short-range communication and long-range communication to one or more networks or to one or more remote devices. Such wireless interfaces may provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 136 may be configured to receive input data from one or more devices, and may also be configured to send output data to other devices.

The non-transitory computer readable medium 132 may include or take the form of memory, such as one or more computer-readable storage media that can be read or accessed by the one or more processor(s) 130. The non-transitory computer readable medium 132 can include volatile and/or non-volatile storage components, such as optical, magnetic, organic or other memory or disc storage, which can be integrated in whole or in part with the one or more processor(s) 130. In some examples, the non-transitory computer readable medium 132 is implemented using a single physical device (e.g., one optical, magnetic, organic or other memory or disc storage unit), while in other examples, the non-transitory computer readable medium 132 is implemented using two or more physical devices. The non-transitory computer readable medium 132 thus is a computer readable storage, and the instructions 134 are stored thereon. The instructions 134 include computer executable code.

The non-transitory computer readable medium 132 also stores the updated cryptographic hash 124 for reference as a password to access the computer system 114.

The one or more processor(s) 130 may be general-purpose processors or special purpose processors (e.g., digital signal processors, application specific integrated circuits, etc.). The one or more processor(s) 130 may receive inputs from the communication interface 136 (e.g., diagnostic test results), and process the inputs to generate outputs that are stored in the non-transitory computer readable medium 132. The one or more processor(s) 130 can be configured to execute the instructions 134 (e.g., computer-readable program instructions) that are stored in the non-transitory computer readable medium 132 and are executable to provide the functionality of the server 102 described herein.

The output interface 138 outputs information for transmission, reporting, or storage, and thus, the output interface 138 may be similar to the communication interface 136 and can be a wireless interface (e.g., transmitter) or a wired interface as well.

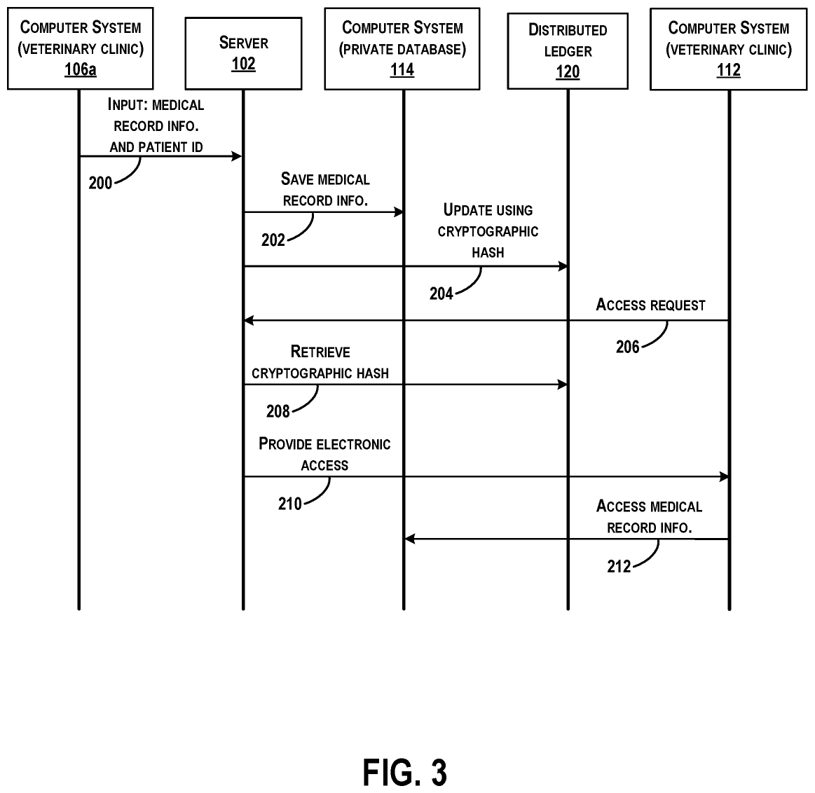

FIG. 3 is an example message diagram illustrating messaging of the system 100 to perform functions of methods described herein, according to one example implementation. Initially, medical record information is created or generated at the veterinary clinic 108a and the computer system 106a sends an input to the server 102 that includes the medical record information associated with a veterinary patient and a veterinary patient identifier unique to the veterinary patient, as shown by message 200.

The medical record information can take many forms. In one example, the medical record information includes diagnostic information such as one or a combination of x-rays, blood test results, veterinarian notes, diagnosis, or other types of medical data, etc. In another example, the medical record information includes a complete medical record for the veterinary patient including insurance and medical information together. In still another example, the medical record information includes information as to ordered/received medication or medical notes. In still further examples, the medical record information includes patient demographic information. In some instances, other data is associated with the medical record information at the computer system 112 including owner information, insurance information, and/or invoice data.

The veterinary patient identifier can also take many forms. In one example, the veterinary patient identifier is a pseudo-random number assigned to the veterinary patient. In another example, the veterinary patient identifier includes patient specific information as well, such as demographic information or information of the animal owner. In another example, the veterinary patient identifier includes any combination of demographic information such as gender, pet type (species), breed, location, age, etc. Importantly, the veterinary patient identifier is unique to the veterinary patient and no two veterinary patient identifiers are the same.

The server 102 then saves the medical record information to the computer system 114 including the privately accessible database, as shown by message 202. The computer system 114 is different from the computer system 106a at the veterinary clinic 108a. The server 102 saves the medical record information to the computer system 114 in a password-protected database. The medical record information is saved and associated with the veterinary patient identifier.

Next, the server 102 accesses the blockchain network 116 to update the distributed ledger 120 to record the save transaction and then associates the veterinary patient identifier with the medical record information saved to the computer system 114 using a cryptographic hash pointing to a data block of the distributed ledger 120, as shown by message 204. The server 102 includes a listing of all assigned veterinary patient identifiers and maintains a mapping of current cryptographic hash codes to each veterinary patient identifier. The server 102 further assigns the current cryptographic hash code as the new password or permissions data required to access the diagnostic information stored at the computer system 114 specific to the veterinary patient associated to the veterinary patient identifier.

In some example, the blockchain network 116 (which may store 512 k of data per block) stores information including the veterinary patient identifier, a practice key (identifier created when a veterinary practice registers with the system and is associated with the veterinary practice), a hash to the medical record information stored off chain at the computer system 114 (which can include a first hash to a results table and a second hash to a treatment table). In one example, a results table includes fields for the veterinary patient identifier, DateofVisit, Practice Key, ResultType (image, note, sign/symptom, test result), ResultValue, ResultUnitMeausure, ServiceCode, ResultID, ResultStatus, LocaleCode, and a link to encrypted unstructured data attachments (notes, images, etc.). In one example, a treatment table includes fields for the veterinary patient identifier, DateofVisit, Practice Key, ReasonForVisit, TreatmentType, TreatmentName, FollowUp, Referral, and Vaccination (if any administered). In further examples, either of the results table and/or the treatment table includes information received from the pet owners, such as over-the-counter medication provided to the veterinary patient or results from a veterinary clinic that is not a participating clinic).

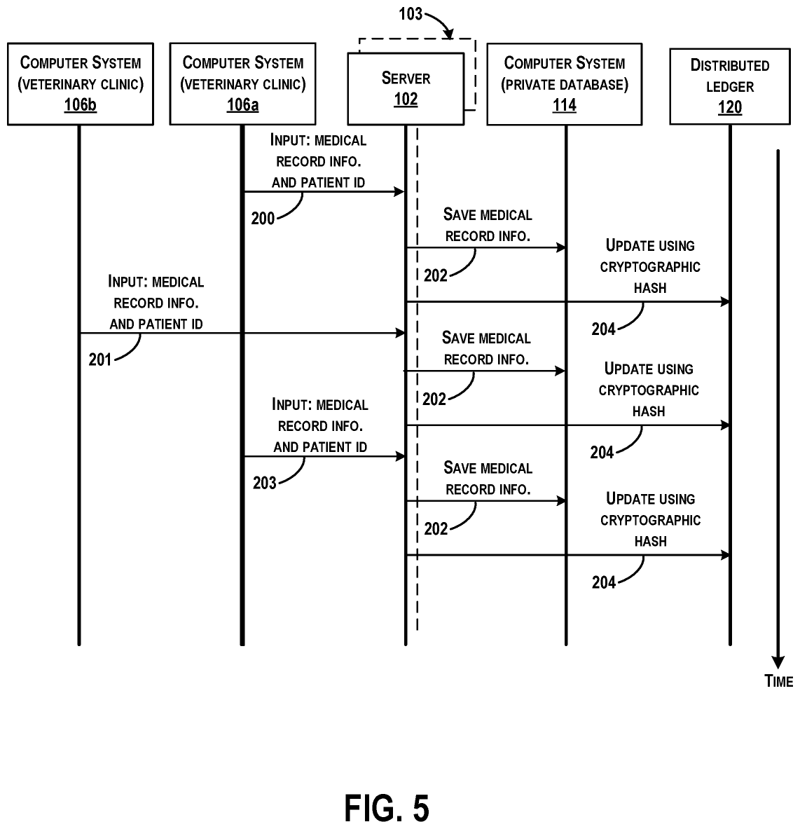

As new medical record information is received for the same veterinary patient identifier, the server 102 aggregates the new additional medical record information for the veterinary patient, which may be received from multiple different computer sources, at the computer system 114. The server 102 then repeats the process of accessing the blockchain network 116 to update the distributed ledger 120 to record the save transaction and then associates the veterinary patient identifier with the additional medical record information saved to the computer system 114 using the updated cryptographic hash pointing to the data block of the distributed ledger 120. Thus, the server 102 always has the updated cryptographic hash mapped to a permissions password required for access to the medical record information stored in the computer system 114, for example. As a result, each time the server 102 saves new medical record information to the computer system 114, the password changes (details described below with respect to FIG. 5).

Following, the server 102 receives an access request, from the computer system 112 located at a different veterinary clinic, that specifies the veterinary patient identifier, as shown by message 206. The server 102 may first verify authenticity of the computer system 112 as a verified clinic.

The server 102 then accesses the distributed ledger 120, using the veterinary patient identifier, to retrieve an updated cryptographic hash, as shown by message 208.

Following, based on the updated cryptographic hash, the server 102 electronically enables access by the computer system 112 to the medical record information of the veterinary patient saved at the computer system 114, as shown by message 210. This may include sending the password to the computer system 112 that includes the updated cryptographic hash. With the updated cryptographic hash, the computer system 112 can now access the medical record information stored at the computer system 114, as shown by message 212. The updated cryptograph hash, for example, is set as the password or permissions data required to access the medical record information for the veterinary patient at the computer system 114.

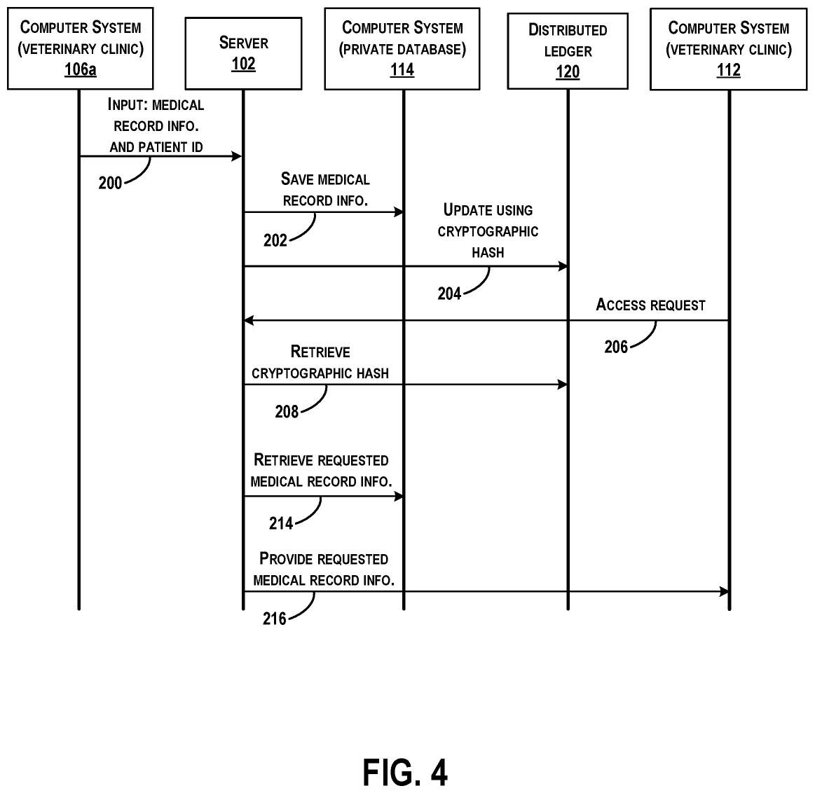

FIG. 4 is another example message diagram illustrating messaging of the system 100 to perform functions of methods described herein, according to one example implementation. In the example shown in FIG. 4, messaging shown by messages 200-208 is the same as messaging described with respect to FIG. 3. However, in the example shown in FIG. 4, the computer system 112 is not given access to or does not directly access the diagnostic data at the computer system 114. Alternatively, after retrieving the updated cryptographic hash, the server 102 retrieves the requested medical record information from the computer system 114, as shown by message 214. The server 102 then provides the requested medical record information to the computer system 112, as shown by message 216.

FIG. 5 is another example message diagram illustrating messaging of the system to perform functions of methods described herein, according to one example implementation. Messaging shown in FIG. 5 relates to changing the password or permissions data required to access the computer system 114, for example.

In FIG. 5, the messages 200, 202, and 204 are the same as described above with respect to FIG. 3. Upon updating the distributed ledger 120 and receiving the updated cryptographic hash, as shown by messages 204, the server 102 changes the password to access the medical record information for the associated veterinary patient identifier to the new updated cryptographic hash. As a result, the medical record information for the associated veterinary patient identifier can only be accessed by first accessing the blockchain network to obtain the current cryptographic hash.

FIG. 5 further illustrates that one of the other computer systems, such as the computer system 106b at the veterinary clinic 108b subsequently sends an input including new medical record information for the veterinary patient identifier to the server 102, as shown by message 201. For example, the pet owner may have taken the veterinary patient to the different veterinary clinic for further testing. Following, the server 102 again saves the medical record information to the computer system 114 and subsequently, the distributed ledger 120 is updated to a new cryptographic hash that is returned to the server 102 and set as the new password to access the most-up-to-date compiled medical record information for the veterinary patient associated with the veterinary patient identifier, as shown by the repeated messages 202 and 204.

This process will continue each time new medical record information is received by the server 102. A third instance is shown by message 203, and the server 102 performs the same save function, and updating the cryptographic hash again to reset the password to access the most-up-to-date compiled medical record information for the veterinary patient associated with the veterinary patient identifier, as shown by the repeated messages 202 and 204.

In FIG. 5, a second server 103 is optionally shown for illustration purposes to note the example in which more than one server is in communication between computer systems at veterinary clinics and the computer system 114 including the privately accessible database. Thus, each server operates to receive medical record information, save the medical record information to the computer system 114, access the blockchain network 116 to receive the updated cryptographic hash, and reset the password at the computer system 114 to access the compiled medical record information for the veterinary patient associated with the veterinary patient identifier to be the updated cryptographic hash.

In sum, the system 100 operates to store medical record information for a veterinary patient at the computer system 114 and associated with a specific veterinary patient identifier assigned to the veterinary patient. The server sets a password at the computer system 114 to access the stored medical record information to be the cryptographic hash obtained from the blockchain network that is created and updated each time a save transaction at the computer system 114 occurs.

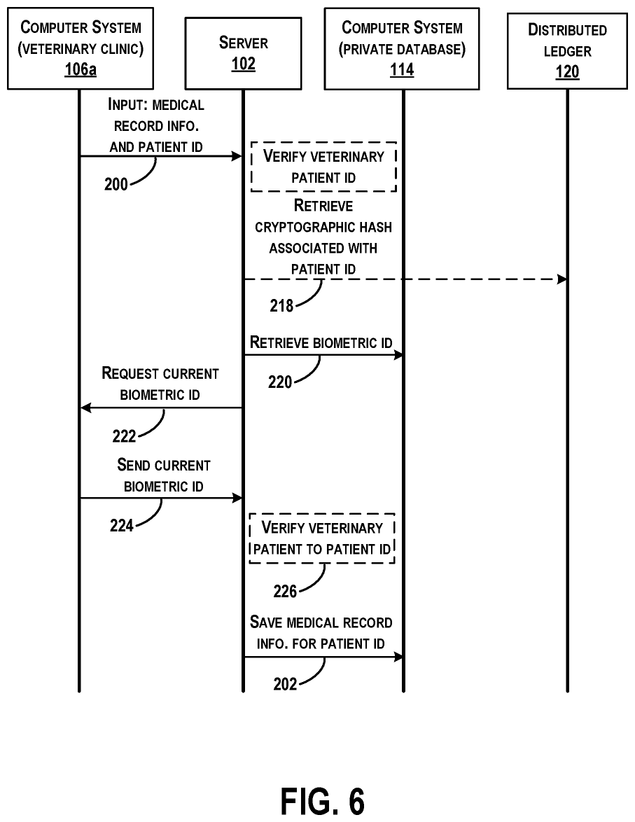

FIG. 6 is another example message diagram illustrating messaging of the system 100 to perform functions of methods described herein, according to one example implementation. Messaging shown in FIG. 6 relates to verifying the veterinary patient identifier, for example.

Initially, again, medical record information is created or generated at the veterinary clinic 108a and the computer system 106a sends an input to the server 102 that includes the medical record information associated with a veterinary patient and a veterinary patient identifier unique to the veterinary patient, as shown by message 200. Following, the server 102 verifies the veterinary patient identifier as being an existing veterinary patient identifier or a valid new veterinary patient identifier. To do so, in one example, the blockchain network 116 can again be accessed to retrieve cryptographic hashes associated with saved transaction noting creation of veterinary patient identifiers, as shown by message 218.

The server 102 then takes steps to authenticate the veterinary patient identifier as being associated with the veterinary patient. To do so, the server 102 retrieves a biometric identifier of the veterinary patient from the computer system 114, as shown by message 220. The server 102 will further request a current biometric identifier from the computer system 106a, as shown by message 222, if the current biometric identifier was not included in the initial input message. The computer system 106a will respond by sending the current biometric identifier to the server 102, and the server 102 then verifies that the veterinary patient is associated with the veterinary patient identifier by matching the biometric identifier to the stored and retrieved biometric identifier of the veterinary patient identifier, as shown at block 226. Once the veterinary patient identifier has been verified and authenticated for the veterinary patient, the server 102 then saves the medical record information to the computer system 114 as previously described and shown by message 202.

The biometric identifier can take many forms. In one example, the biometric identifier of the veterinary patient includes a genetic sequence of the veterinary patient. In this example, the medical record information includes testing data or samples of the veterinary patient from which the genetic sequence is obtainable.

In another example, the biometric identifier of the veterinary patient includes a nose-print of the veterinary patient. The nose-print of an animal is similar to a fingerprint of a human, and is unique to each animal.

In another example, the biometric identifier of the veterinary patient including a voice-print of the veterinary patient. Like a nose-print, a voice-print is unique to each animal.

In another example, the biometric identifier of the veterinary patient includes an image of the veterinary patient. The image can be updated over time and used to validate the veterinary patient to the veterinary patient identifier.

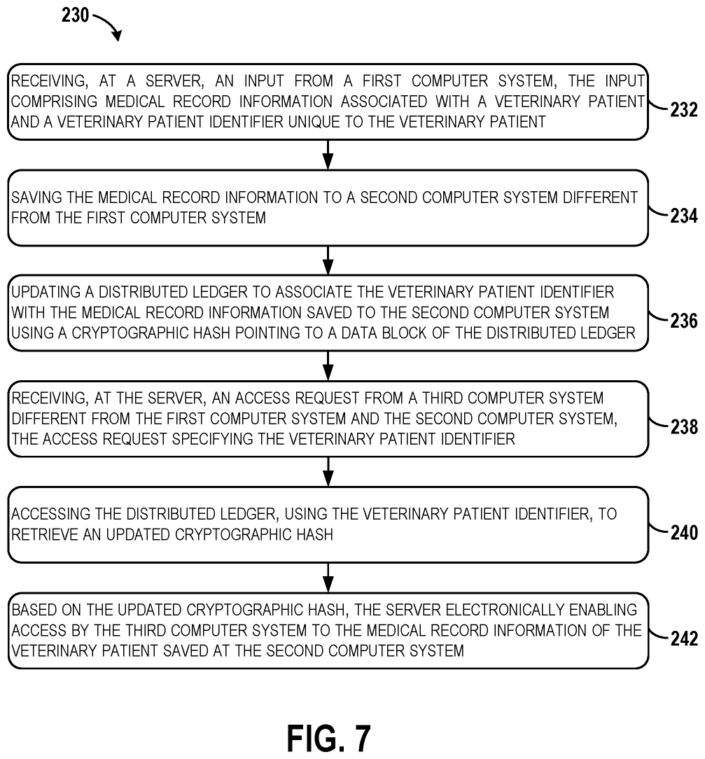

FIG. 7 shows a flowchart of an example of a method 230 for enabling electronic access to medical record information of a veterinary patient over a network, according to an example implementation. Method 230 shown in FIG. 7 presents an example of a method that could be used with the system 100 shown in FIG. 1 or the server 102 shown in FIG. 2, for example. Further, devices or systems may be used or configured to perform logical functions presented in FIG. 7. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 230 may include one or more operations, functions, or actions as illustrated by one or more of blocks 232-242. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

Thus, in an example operation, each block or portions of each block 232-242 of the method 230 takes the form of the instructions 134 stored in the non-transitory computer readable medium 132 of the server 102, which when executed by the one or more processor(s) 130, causes the one or more processor(s) 130 to perform the stated functions of the blocks 232-242.

In addition, each block or portions of each block in FIG. 7, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 232, the method 230 includes receiving, at the server 102, an input from a first computer system (e.g., the computer system 106a), and the input comprises medical record information associated with a veterinary patient and a veterinary patient identifier unique to the veterinary patient. In some examples, the first computer system is not the first entity to see the veterinary patient, and reference to “first” does not require first in time, for example.

At block 234, the method 230 includes saving the medical record information to a second computer system (e.g., the computer system 114) different from the first computer system (e.g., the computer system 106a). In one example, saving the medical record information to the second computer system comprises saving the medical record information to a password-protected database.

At block 236, the method 230 includes updating a distributed ledger to associate the veterinary patient identifier with the medical record information saved to the second computer system using a cryptographic hash pointing to a data block of the distributed ledger. In one example, the method 230 also includes aggregating additional medical record information for the veterinary patient received from multiple different computer sources at the second computer system, and then again updating the distributed ledger to associate the veterinary patient identifier with the additional medical record information saved to the second computer system using a second updated cryptographic hash pointing to the data block of the distributed ledger.

In one example, updating the distributed ledger comprises updating a public blockchain, and the public blockchain is a decentralized network with open access rights. In another example, updating the distributed ledger comprises updating a private blockchain, and the private blockchain is a permission-based network accessible by invitation.

At block 238, the method 230 includes receiving, at the server 102, an access request from a third computer system (e.g., the computer system 112) different from the first computer system (e.g., the computer system 106a) and the second computer system (e.g., the computer system 114), and the access request specifies the veterinary patient identifier. The first and third computer systems may reside at the same veterinary clinic in some examples (such as one computer system for in-house data and one for reference lab data).

In one example, the input is received from the first computer system residing at a first veterinary clinic located at a first location, and the access request is received from the third computer system residing at a second veterinary clinic located at a second location different from the first location.

At block 240, the method 230 includes accessing the distributed ledger, using the veterinary patient identifier, to retrieve an updated cryptographic hash.

At block 242, the method 230 includes based on the updated cryptographic hash, the server electronically enabling access by the third computer system to the medical record information of the veterinary patient saved at the second computer system.

In further examples, the method 230 optionally includes receiving a biometric identifier of the veterinary patient, and verifying that the veterinary patient is associated with the veterinary patient identifier by the server matching the biometric identifier to a stored biometric identifier of the veterinary patient identifier. In one example, the function of receiving the biometric identifier of the veterinary patient comprises processing the medical record information to identify a genetic sequence. In other examples, the function of receiving the biometric identifier of the veterinary patient comprises any one or combination of receiving a nose-print of the veterinary patient, receiving a voice-print of the veterinary patient, or receiving an image of the veterinary patient.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination or any sub-combination, and all of such possibilities are intended to be within the scope of the disclosure.

Thus, examples of the present disclosure relate to enumerated clauses (ECs) listed below in any combination or any sub-combination.

EC 1 is a computer-implemented method for enabling electronic access to medical record information of a veterinary patient over a network that comprises receiving, at a server, an input from a first computer system, the input comprising medical record information associated with a veterinary patient and a veterinary patient identifier unique to the veterinary patient; saving the medical record information to a second computer system different from the first computer system; updating a distributed ledger to associate the veterinary patient identifier with the medical record information saved to the second computer system using a cryptographic hash pointing to a data block of the distributed ledger; receiving, at the server, an access request from a third computer system different from the first computer system and the second computer system, the access request specifying the veterinary patient identifier; accessing the distributed ledger, using the veterinary patient identifier, to retrieve an updated cryptographic hash; and based on the updated cryptographic hash, the server electronically enabling access by the third computer system to the medical record information of the veterinary patient saved at the second computer system.

EC 2 is the method of EC 1, wherein: receiving the input from the first computer system comprises receiving the input from a first veterinary clinic located at a first location; and receiving the access request from the third computer system comprises receiving the access request from a second veterinary clinic located at a second location different from the first location.

EC 3 is the method of any of ECs 1-2, wherein: saving the medical record information to the second computer system comprises saving the medical record information to a password-protected database.

EC 4 is the method of any of ECs 1-3, wherein: updating the distributed ledger comprises updating a public blockchain, wherein the public blockchain is a decentralized network with open access rights.

EC 5 is the method of any of ECs 1-3, wherein: updating the distributed ledger comprises updating a private blockchain, wherein the private blockchain is a permission-based network accessible by invitation.

EC 6 is the method of any of ECs 1-5, further comprising: aggregating additional medical record information for the veterinary patient received from multiple different computer sources at the second computer system; and updating the distributed ledger to associate the veterinary patient identifier with the additional medical record information saved to the second computer system using a second updated cryptographic hash pointing to the data block of the distributed ledger.

EC 7 is the method of any of ECs 1-6, further comprising: receiving a biometric identifier of the veterinary patient; and verifying that the veterinary patient is associated with the veterinary patient identifier by the server matching the biometric identifier to a stored biometric identifier of the veterinary patient identifier.

EC 8 is the method of any of ECs 1-7, wherein: receiving the biometric identifier of the veterinary patient comprises processing the medical record information to identify a genetic sequence.

EC 9 is the method of any of ECs 1-7, wherein: receiving the biometric identifier of the veterinary patient comprises receiving a nose-print of the veterinary patient.

EC 10 is the method of any of ECs 1-7, wherein: receiving the biometric identifier of the veterinary patient comprises receiving a voice-print of the veterinary patient.

EC 11 is the method of any of ECs 1-7, wherein: receiving the biometric identifier of the veterinary patient comprises receiving an image of the veterinary patient.

EC 12 is a server comprising: one or more processors; and non-transitory computer readable medium having stored therein instructions that when executed by the one or more processors, causes the server to perform functions comprising: receiving an input from a first computer system, the input comprising medical record information associated with a veterinary patient and a veterinary patient identifier unique to the veterinary patient; saving the medical record information to a second computer system different from the first computer system; updating a distributed ledger to associate the veterinary patient identifier with the medical record information saved to the second computer system using a cryptographic hash pointing to a data block of the distributed ledger; receiving an access request from a third computer system different from the first computer system and the second computer system, the access request specifying the veterinary patient identifier; accessing the distributed ledger, using the veterinary patient identifier, to retrieve an updated cryptographic hash; and based on the updated cryptographic hash, the server electronically enabling access by the third computer system to the medical record information of the veterinary patient saved at the second computer system.

EC 13 is the server of EC 12, wherein: receiving the input from the first computer system comprises receiving the input from a first veterinary clinic located at a first location; and receiving the access request from the third computer system comprises receiving the access request from a second veterinary clinic located at a second location different from the first location.

EC 14 is the server of any of ECs 12-13, wherein: saving the medical record information to the second computer system comprises saving the medical record information to a password-protected database.

EC 15 is the server of any of ECs 12-14, wherein: updating the distributed ledger comprises updating a public blockchain, wherein the public blockchain is a decentralized network with open access rights.

EC 16 is the server of any of ECs 12-14, wherein: updating the distributed ledger comprises updating a private blockchain, wherein the private blockchain is a permission-based network accessible by invitation.

EC 17 is the server of any of ECs 12-16, wherein the functions further comprise: aggregating additional medical record information for the veterinary patient received from multiple different computer sources at the second computer system; and updating the distributed ledger to associate the veterinary patient identifier with the additional medical record information saved to the second computer system using a second updated cryptographic hash pointing to the data block of the distributed ledger.

EC 18 is the server of any of ECs 12-17, wherein the functions further comprise: receiving a biometric identifier of the veterinary patient; and verifying that the veterinary patient is associated with the veterinary patient identifier by the server matching the biometric identifier to a stored biometric identifier of the veterinary patient identifier.

EC 19 is a non-transitory computer readable medium having stored thereon instructions, that when executed by one or more processors of a computing device, cause the computing device to perform functions comprising: receiving an input from a first computer system, the input comprising medical record information associated with a veterinary patient and a veterinary patient identifier unique to the veterinary patient; saving the medical record information to a second computer system different from the first computer system; updating a distributed ledger to associate the veterinary patient identifier with the medical record information saved to the second computer system using a cryptographic hash pointing to a data block of the distributed ledger; receiving an access request from a third computer system different from the first computer system and the second computer system, the access request specifying the veterinary patient identifier; accessing the distributed ledger, using the veterinary patient identifier, to retrieve an updated cryptographic hash; and based on the updated cryptographic hash, the server electronically enabling access by the third computer system to the medical record information of the veterinary patient saved at the second computer system.

EC 20 is the non-transitory computer readable medium of EC 19, wherein: receiving the input from the first computer system comprises receiving the input from a first veterinary clinic located at a first location; and receiving the access request from the third computer system comprises receiving the access request from a second veterinary clinic located at a second location different from the first location.

EC 21 is the non-transitory computer readable medium of any of ECs 19-20, wherein the functions further comprise: aggregating additional medical record information for the veterinary patient received from multiple different computer sources at the second computer system; and updating the distributed ledger to associate the veterinary patient identifier with the additional medical record information saved to the second computer system using a second updated cryptographic hash pointing to the data block of the distributed ledger.

EC 22 is the non-transitory computer readable medium of any of ECs 19-21, wherein the functions further comprise: receiving a biometric identifier of the veterinary patient; and verifying that the veterinary patient is associated with the veterinary patient identifier by the server matching the biometric identifier to a stored biometric identifier of the veterinary patient identifier.

By the term “substantially” and “about” used herein, it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide. The terms “substantially” and “about” represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A computer-implemented method for enabling electronic access to medical record information of a veterinary patient over a network, the method comprising:
receiving, at a server, an input from a first computer system, the input comprising medical record information associated with a veterinary patient and a veterinary patient identifier unique to the veterinary patient;
saving the medical record information to a second computer system different from the first computer system;
updating a distributed ledger to associate the veterinary patient identifier with the medical record information saved to the second computer system using a cryptographic hash pointing to a data block of the distributed ledger;
receiving, at the server, an access request from a third computer system different from the first computer system and the second computer system, the access request specifying the veterinary patient identifier;
accessing the distributed ledger, using the veterinary patient identifier, to retrieve an updated cryptographic hash; and
based on the updated cryptographic hash, the server electronically enabling access by the third computer system to the medical record information of the veterinary patient saved at the second computer system.

2. The computer-implemented method of claim 1, wherein:
receiving the input from the first computer system comprises receiving the input from a first veterinary clinic located at a first location; and
receiving the access request from the third computer system comprises receiving the access request from a second veterinary clinic located at a second location different from the first location.

3. The computer-implemented method of claim 1, wherein:
saving the medical record information to the second computer system comprises saving the medical record information to a password-protected database.

4. The computer-implemented method of claim 1, wherein:
updating the distributed ledger comprises updating a public blockchain, wherein the public blockchain is a decentralized network with open access rights.

5. The computer-implemented method of claim 1, wherein:
updating the distributed ledger comprises updating a private blockchain, wherein the private blockchain is a permission-based network accessible by invitation.

6. The computer-implemented method of claim 1, further comprising:
aggregating additional medical record information for the veterinary patient received from multiple different computer sources at the second computer system; and
updating the distributed ledger to associate the veterinary patient identifier with the additional medical record information saved to the second computer system using a second updated cryptographic hash pointing to the data block of the distributed ledger.

7. The computer-implemented method of claim 1, further comprising:
receiving a biometric identifier of the veterinary patient; and
verifying that the veterinary patient is associated with the veterinary patient identifier by the server matching the biometric identifier to a stored biometric identifier of the veterinary patient identifier.

8. The computer-implemented method of claim 7, wherein:
receiving the biometric identifier of the veterinary patient comprises processing the medical record information to identify a genetic sequence.

9. The computer-implemented method of claim 7, wherein:
receiving the biometric identifier of the veterinary patient comprises receiving a nose-print of the veterinary patient.

10. The computer-implemented method of claim 7, wherein:
receiving the biometric identifier of the veterinary patient comprises receiving a voice-print of the veterinary patient.

11. The computer-implemented method of claim 7, wherein:
receiving the biometric identifier of the veterinary patient comprises receiving an image of the veterinary patient.

12. A server comprising:
one or more processors; and
non-transitory computer readable medium having stored therein instructions that when executed by the one or more processors, causes the server to perform functions comprising:
receiving an input from a first computer system, the input comprising medical record information associated with a veterinary patient and a veterinary patient identifier unique to the veterinary patient;
saving the medical record information to a second computer system different from the first computer system;
updating a distributed ledger to associate the veterinary patient identifier with the medical record information saved to the second computer system using a cryptographic hash pointing to a data block of the distributed ledger;
receiving an access request from a third computer system different from the first computer system and the second computer system, the access request specifying the veterinary patient identifier;
accessing the distributed ledger, using the veterinary patient identifier, to retrieve an updated cryptographic hash; and
based on the updated cryptographic hash, electronically enabling access by the third computer system to the medical record information of the veterinary patient saved at the second computer system.

13. The server of claim 12, wherein:
receiving the input from the first computer system comprises receiving the input from a first veterinary clinic located at a first location; and
receiving the access request from the third computer system comprises receiving the access request from a second veterinary clinic located at a second location different from the first location.

14. The server of claim 12, wherein:
saving the medical record information to the second computer system comprises saving the medical record information to a password-protected database.

15. The server of claim 12, wherein:
updating the distributed ledger comprises updating a public blockchain, wherein the public blockchain is a decentralized network with open access rights.

16. The server of claim 12, wherein:
updating the distributed ledger comprises updating a private blockchain, wherein the private blockchain is a permission-based network accessible by invitation.

17. The server of claim 12, wherein the functions further comprise:
aggregating additional medical record information for the veterinary patient received from multiple different computer sources at the second computer system; and
updating the distributed ledger to associate the veterinary patient identifier with the additional medical record information saved to the second computer system using a second updated cryptographic hash pointing to the data block of the distributed ledger.

18. The server of claim 12, wherein the functions further comprise:
receiving a biometric identifier of the veterinary patient; and
verifying that the veterinary patient is associated with the veterinary patient identifier by the server matching the biometric identifier to a stored biometric identifier of the veterinary patient identifier.

19. A non-transitory computer readable medium having stored thereon instructions, that when executed by one or more processors of a computing device, cause the computing device to perform functions comprising:
receiving an input from a first computer system, the input comprising medical record information associated with a veterinary patient and a veterinary patient identifier unique to the veterinary patient;
saving the medical record information to a second computer system different from the first computer system;
updating a distributed ledger to associate the veterinary patient identifier with the medical record information saved to the second computer system using a cryptographic hash pointing to a data block of the distributed ledger;
receiving an access request from a third computer system different from the first computer system and the second computer system, the access request specifying the veterinary patient identifier;
accessing the distributed ledger, using the veterinary patient identifier, to retrieve an updated cryptographic hash; and
based on the updated cryptographic hash, electronically enabling access by the third computer system to the medical record information of the veterinary patient saved at the second computer system.

20. The non-transitory computer readable medium of claim 19, wherein:
receiving the input from the first computer system comprises receiving the input from a first veterinary clinic located at a first location; and
receiving the access request from the third computer system comprises receiving the access request from a second veterinary clinic located at a second location different from the first location.

21. The non-transitory computer readable medium of claim 19, wherein the functions further comprise:
aggregating additional medical record information for the veterinary patient received from multiple different computer sources at the second computer system; and
updating the distributed ledger to associate the veterinary patient identifier with the additional medical record information saved to the second computer system using a second updated cryptographic hash pointing to the data block of the distributed ledger.

22. The non-transitory computer readable medium of claim 19, wherein the functions further comprise:
receiving a biometric identifier of the veterinary patient; and
verifying that the veterinary patient is associated with the veterinary patient identifier by matching the biometric identifier to a stored biometric identifier of the veterinary patient identifier.
